(19) United States
(12) Patent Application Publication
(10) Pub. No.: US 2024/0398839 A1
(43) Pub. Date: Dec. 5, 2024

(54) CANNABINOID-LIPID CONJUGATES, METHODS FOR PRODUCING THE SAME AND USES THEREOF

(71) Applicant: RAMOT AT TEL-AVIV UNIVERSITY LTD., Tel Aviv (IL)

(72) Inventors: Daniel OFFEN, Tel Aviv (IL); Nataly YOM-TOV, Tel Aviv (IL)

(73) Assignee: RAMOT AT TEL-AVIV UNIVERSITY LTD., Tel Aviv (IL)

(21) Appl. No.: 18/694,614

(22) PCT Filed: Sep. 22, 2022
(86) PCT No.: PCT/IL2022/051012

§ 371 Date: Mar. 22, 2024

(30) Foreign Application Priority Data
Sep. 22, 2021 (IL) 286604

(51) Int. Cl. A61K 31/00; A61K 47/54

(52) U.S. Cl. CPC A61K 31/658; A61K 47/544

(57) ABSTRACT

Provided is a cannabinoid-phospholipid conjugate where the cannabinoid is covalently linked, via a cleavable linker, to a polar head group of the phospholipid. Also provided is a method of obtaining the conjugate, the method comprising (a) reacting the phospholipid (PL) dissolved in an organic solvent with maleic anhydride (MA) to obtain PL-MA intermediate; and (b) reacting said PL-MA intermediate with the cannabinoid in the presence of a carboxyl activating agent and an esterification agent to obtain a reaction mixture comprising said conjugate. Uses of the conjugate are also disclosed.

### TECHNOLOGICAL FIELD

The present disclosure concerns cannabinoid-lipid conjugates.

### BACKGROUND ART

References considered to be relevant as background to the presently disclosed subject matter are listed below:
International Patent Application Publication No. WO2017/106957
International Patent Application Publication No. WO2019/200043
International Patent Application Publication No. WO202191477
International Patent Application Publication No. WO2021/184010

Acknowledgement of the above references herein is not to be inferred as meaning that these are in any way relevant to the patentability of the presently disclosed subject matter.

### BACKGROUND

WO2017/106957 describes lipid linked pro-drugs and their therapeutic use.

WO2019/200043 describes lipid prodrugs that self-assemble into lipid microbubbles or liposomes. The prodrug-loaded microbubbles or liposomes can be active intracellularly using an external stimulus, for example, ultrasound waves.

WO202191477 describes lipid conjugates for the delivery of a molecule of interest such as a drug moiety, the conjugate comprising a linker group such as ester, ether or carbamate. The lipid conjugate can be formulated in a drug delivery vehicle such as a lipid nanoparticle (LNP).

WO2021/184010 describes nano-formulations of cannabidiol (CBD) and other cannabinoids as well as method of treating specific ocular diseases using the nano-formulations.

### GENERAL DESCRIPTION

The present disclosure provides, in accordance with a first aspect of the presently disclosed subject matter, a cannabinoid-phospholipid conjugate having the general formula (I):

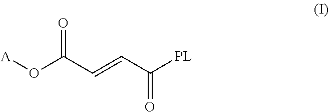

wherein A is a cannabinoid and PL is a phospholipid (PL); and
wherein the cannabinoid (A) is covalently linked, via a cleavable linker, to a polar head group of the PL.

In some examples, the cannabinoid A is linked to the cleavable linker at a position occupied by a hydroxyl group when said cannabinoid A is in free form.

Also provided by a second aspect of the presently disclosed subject matter, is a method for obtaining a cannabinoid-phospholipid conjugate having the general formula (I):

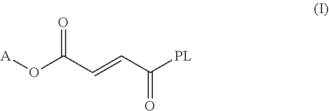

wherein A is a cannabinoid and PL is a phospholipid (PL); and
wherein the cannabinoid (A) is covalently linked, via a cleavable linker, to a polar head group of the PL.

In some examples, the cannabinoid A is linked to the cleavable linker through an oxygen bridge at a position occupied in the cannabinoid by a hydroxyl group when said cannabinoid A is in free form; the method comprises, in its broadest scope, reacting a phospholipid (PL) with the cannabinoid (A) in a reaction method involving the formation of an intermediate conjugate with maleic anhydride.

More specifically, the method comprises:
(i) reacting the phospholipid (PL), dissolved in an organic solvent system, with maleic anhydride (MA), to obtain PL-MA intermediate;
(ii) reacting said PL-MA intermediate with the cannabinoid in the presence of a carboxyl activating agent and an esterification agent to obtain a reaction mixture comprising said conjugate.

Also provided in accordance with a further aspect of the presently disclosed subject matter is a cannabinoid-phospholipid conjugate as disclosed herein, for use as a vehicle for releasing said cannabinoid, in free form, at a target site.

Finally, there is provided, in accordance with yet a further aspect of the presently disclosed subject matter, a method for delivering a cannabinoid to a target site in a subject in need thereof, the method comprises administering to said subject an amount of a cannabinoid-phospholipid conjugate as disclosed herein.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a Liquid Chromatography Mass Spectroscopies of the exemplary intermediate DLPE-MA, showing the intermediate peak at 4.119 minutes.

FIGS. 2A-2C provide Nuclear Magnetic Resonance (NMR) spectroscopies of the non-limiting exemplary final DLPE-MA-CBD conjugate of, respectively, the ¹H-NMR (FIG. 2A), ¹³C-NMR (FIG. 2B) and ³¹P-NMR (FIG. 2C).

FIGS. 3A-3C provide high pressure liquid chromatogram (HPLC) (FIG. 3A), UV absorbance spectrum (FIG. 3B) and Nuclear Magnetic Resonance (¹H-NMR, 400 MHz, FIG. 3C) of exemplary final DLPE-MA-CBD conjugate according to non-limiting Example 1B.

FIGS. 4A-4B provide a computational presentation of DLPE-MA-CBD conjugate in accordance with an example of the present disclosure, illustrating the phosphorous group to which the two fatty acid chains are linked, the cleavable linker and the CBD group (FIG. 4A) and the spatial configuration of the conjugate, similarly pointing at the phosphorous group to which the two fatty acid chains are linked, the cleavable linker and the CBD group (FIG. 4B).

FIGS. 5A-5C provide high pressure liquid chromatogram (HPLC) (FIG. 5A), UV absorbance spectrum (FIG. 5B) and Nuclear Magnetic Resonance (¹H-NMR, 400 MHz, FIG. 5C) of exemplary final DLPE-MA-CBN conjugate according to non-limiting Example 2.

### DETAILED DESCRIPTION

The presently disclosed subject matter is based on the identification of a cleavable, yet covalent, linkage between a lipid moiety and a cannabidiol that allows for the enzymatic release of the cannabidiol once within the suitable environment (e.g. within a living body).

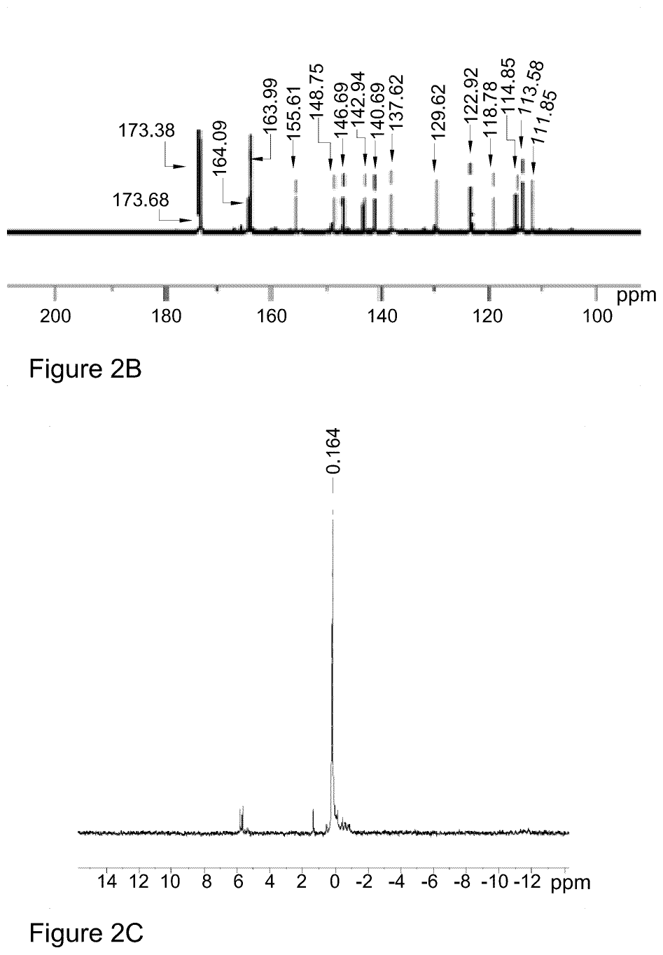

Specifically, it has been found that a conjugation a lipid moiety to one of the cannabidiol hydroxyl groups (when the cannabidiol is unbound to any moiety), via a maleic acid linkage (“linker”), provides a unique spatial configuration, as illustrated in the non-limiting example of FIGS. 2A-2B, that permits access to enzymatic cleavage and release of the cannabinoid, once brought into proximity with the enzyme.

Thus, in accordance with a first aspect of the presently disclosed subject matter, there is provided a cannabinoid-phospholipid conjugate having the general formula (I):

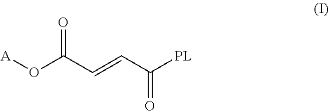

wherein A is a cannabinoid and PL is a phospholipid (PL); and
the cannabinoid (A) is covalently linked, via a cleavable linker, to a polar head group of the PL.

In some examples, as noted above, the cannabinoid A is linked to the cleavable linker at a position occupied by a hydroxyl group present in the cannabinoid, when the cannabinoid A is in free form.

As used herein the term “cannabinoid” refers to a chemical substance, preferably low molecular weight compound, that shows direct or indirect activity on the endocannabinoid system, e.g., to induce receptors and downstream effectors of the endocannabinoid system. It is to be understood that the term “cannabinoid” as defined herein includes but is not limited to purified food and pharmaceutical grade substances, which may be obtained by purification from a natural source or via synthetic means. The cannabinoid may be a purified isolated individual cannabinoids, synthetic cannabinoids and analogues thereof, cannabidiol (CBD) or analogues thereof, tetrahydrocannabinol (THC) or analogues thereof.

In some examples of the presently disclosed subject matter, a low molecular weight compound is one having a weight of equal or less than 1,000 kDa.

In some examples of the presently disclosed subject matter, the cannabinoid is a cannabidiol (2-[(1R,6R)-6-Isopropenyl-3-methylcyclohex-2-en-1-yl]-5-pentylbenzene-1,3-diol, also known by the abbreviated name “CBD”) or a CBD functional analogue thereof.

When referring to a “functional analogue” it is to be understood to include any compound (preferably low molecular weight compound) that binds to a cannabidiol receptor, with either a same or a greater potency as compared to the respective natural cannabinoid, to which it is analogous. In some examples, the functional analogue also shares a degree of structural similarity with respective natural cannabinoid.

For simplicity, the term “CBD” is used herein to collectively refer to the naturally occurring CBD as well as to CBD analogues (synthetic or semi synthetic). When intending to refer specifically to the naturally occurring CBD, namely to (2-[(1R,6R)-6-Isopropenyl-3-methylcyclohex-2-en-1-yl]-5-pentylbenzene-1,3-diol, the term “natural CBD” will be used.

Examples of cannabinoids include, without being limited thereto, CBD, the synthetic Cannabidiol-dimethylheptyl (CBD-DMH), the phytocannabinoids Cannabidivarin (CBDV), Cannabidivarinolic acid (CBDVA), cannabidiolic acid (CBDA), Cannabidiol monomethyl ether (CBDM), cannabidiolquinones (CBDQ), Cannabidiol hydroxy quinone (CBDHQ), and abnormal CBD (Abn-CBD) [Paula Morales, Patricia H. Reggio, and Nadine Jagerovic “An Overview on Medicinal Chemistry of Synthetic and Natural Derivatives of Cannabidiol” Front Pharmacol.” 8:422, (2017)], quinone of CBD [See for example, Natalya M. Kogan, Maximilian Peters, Raphael Mechoulam-Cannabinoid Quinones-A Review and Novel Observations Molecules 2021; and Kogan NM, Peters M, Mechoulam R. Cannabinoid Quinones-A Review and Novel Observations. Molecules. 2021 Mar. 21;26 (6): 1761. doi: 10.3390/molecules26061761. PMID: 33801057; PMCID: PMC8003933, the content of both with respect to abnormal cannabinoids, being incorporated herein, in their entirety, by reference].

In some examples, the cannabinoid is the natural CBD.

In some examples, the cannabinoid is a tetrahydrocannabinol (THC) including also known by the abbreviated name “delta9-THC”, “delta8-THC” or “THC”) or a THC functional analogue thereof.

For simplicity, the term “THC” is used herein to collectively refer to the naturally occurring THC as well as to THC analogues (including synthetic or semi synthetic). When intending to refer specifically to the naturally occurring THC, the term “delta9-THC” will be used.

Examples of THC include, without being limited thereto, include delta9-THC, delta8-THC, trans-DELTA10-tetrahydrocannabinol (trans-DELTA10-THC), cis-DIO-tetrahydrocannabinol (cis-DELTA10-THC), tetrahydrocannabinolic acid C4 (THCA-C4), tetrahydrocannbinol C4 (THC-C4), tetrahydrocannabivarinic acid (THCVA), tetrahydrocannabivarin (THCV), DELTA8-tetrahydrocannabivarin (DELTA8-THCV), DELTA9-tetrahydrocannabivarin (DELTA9-THCV), Delta-9-tetrahydrocannabinolic acid B (DELTA9-THCA-B), tetrahydrocannabiorcolic acid (THCA-C1), tetrahydrocannabiorcol (THC-C1), DELTA7-cis-iso-tetrahydrocannabivarin, DELTA8-tetrahydrocannabinolic acid (DELTA8-THC-A), DELTA9-tetrahydrocannabinolic acid (DELTA9-THC-A), 11-hydroxy-DELTA9-tetrahydrocannabinol (11-OH-THC), 11-nor-9-carboxy-DELTA9-tetrahydrocannabinol, 10 ethoxy-9-hydroxy-DELTA6a-tetrahydrocannabinol, 10-oxo-DELTA6a (10a)-tetrahydrocannabinol (OTHC), DELTA9-cis-tetrahydrocannabinol (cis-THC), trihydroxy-delta-9-tetrahydrocannabinol (triOH-THC), Delta-9-tetrahydrocannabivarinic acid (THCVA), 10-Oxo-delta-6a-tetrahydrocannabinol (OTHC), isotetrahydrocannabinol (iso-THC).

In some examples, the cannabinoid is the delta9-THC.

In some examples, the cannabinoid is a cannabigerol (CBG) or a functional analogue thereof.

In some examples, the CBG or functional analogue thereof is selected from the group consisting of cannabigerol (CBG), cannabigerolic acid (CBGA), cannabigerovarinic acid (CBGVA), cannabigerol monomethyl ether (CBGM), cannabigerovarinic acid (CBGVA), cannabigerolic acid monomethylether (CBGAM), cannabigerovarin (CBGV), and quinone of CBG [Kogan NM, Peters M, Mechoulam R. Cannabinoid Quinones-A Review and Novel Observations. Molecules. 2021 Mar. 21: 26 (6): 1761. doi: 10.3390/molecules26061761. PMID: 33801057; PMCID: PMC8003933].

Other cannabinoids that fall within the scope of the presently disclosed subject matter include, without being limited thereto, cannabichromene (CBC), cannabichromanone (CBCN), cannabichromenic acid (CBCA), cannabivarichromene (CBCV), cannabichromevarinic acid (CBCVA), cannabinol (CBN), cannabinolic acid (CBNA), cannabinol methyl ether (CBNM), cannabinol C4 (CBN-C4), cannabinol C2 (CBN-C2), cannabinol C1 (CBN-C1), cannabinodiol (CBND), cannabielsoin (CBE), cannabielsoic acid A (CBEA-A), Cannabielsoic acid B (CBEA-B), cannabicyclol (CBL), cannabicycloic acid (CBLA), cannabicyclovarin (CBLV), cannabitriol (CBT), cannabitriolvarin (CBTV), ethoxy-cannabitriolvarin (CBTVE), cannabivarin (CBV), cannabinodivarin (CBVD), tetrahydrocannabivarin (THCV), cannabifuran (CBF), dehydrocannabifuran (DCBF), cannabirispol (CBR), each constituting a separate embodiment of the present disclosure.

In some examples, the cannabinoid within the conjugate is CBN.

The cannabinoid is linked, via a linker, to a phospholipid (PL).

In the context of the presently disclosed subject matter, when referring to a phospholipid it is to be understood to encompass any member of lipids having a glycerol backbone (glycerophospholipids, GPLs), a sphingosine-backbone (SPLs) or an alkyl-phospholipid backbone (Alkyl-GPLs) each having at least one fatty acid linked with an ether bond at the sn-1 of the glycerol backbone. Further, linked to the backbone there is a phosphate carrying a polar headgroup.

In some examples of the presently disclosed subject matter, the phospholipid can be represented by the general formula (II):

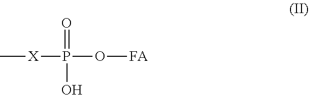

wherein
FA represents a glycerol backbone or a sphinogosine backbone carrying one or two acyl, alkyl or alkenyl chains, which may be the same or different;
X represents an oxygen or a hydrophilic head group to which said cleavable linker is bound (not illustrated in formula II).

In some examples of the presently disclosed subject matter, the phospholipid is a glycerophospholipid, namely, FA comprises a glycerol backbone or a sphingosine.

In some examples of the presently disclosed subject matter, FA is a glycerol backbone.

In some examples of the presently disclosed subject matter, X is oxygen to which the cleavable linker is bound.

In some examples of the presently disclosed subject matter, X is the phospholipid polar headgroup to which the cleavable linker is bound.

When referring to glycerophospholipids at least one, preferably two of the hydroxyl groups of the glycerol backbone is substituted by, respectively, one or two of an acyl, alkyl or alkenyl chains and the third hydroxyl group of the glycerol backbone is substituted by a phosphate group carrying a polar headgroup.

In some examples, the acyl, alkyl or alkenyl chains are typically between about 6 and about 24 carbon atoms in length, at times, between about 8 and about 24 carbon atoms in length; or at times between about 10 and about 24 carbon atoms in length; or at times, between about 12 and about 24 carbon atoms in length, and have varying degrees of saturation being fully, partially or non-hydrogenated chains.

The cannabinoid is linked to the polar headgroup of the phospholipid via a linking portion/segment, referred to herein as the linker portion. To allow this linkage, the polar headgroup of the phospholipid is one that is capable of, according to some examples of the present disclosure, reacting with maleic anhydride (MA).

In some examples of the presently disclosed subject matter, the reaction between the cannabinoid and the polar headgroup of the PL can be in the presence of a base, including organic base, e.g. pyridine and/or inorganic base, as further described below.

Without being limited thereto, the polar headgroup is selected from the group consisting of serine (phosphatidylserine, PS), ethanolamine (phosphatidylethanolamine, PE), inositol (phosphatidylinositol, PI), glycerol (phosphatidylglycerol, PG).

In some examples of the presently disclosed subject matter, the phospholipid has a glycerol backbone to which C10-C24 fatty acids (which may be the same or different) are bound to the sn-1 and sn-2 positions.

In some examples of the presently disclosed subject matter, the phospholipid is 1,2-Dilauroyl-sn-glycero-3-phosphorylethanolamine namely, a glycerol backbone comprising a medium chain (12:0) lauric acid at the sn-1 and sn-2 positions, and the phosphorylethanolamine at the sn-3 position.

In some examples of the presently disclosed subject matter, the polar headgroup is ethanolamine, i.e. the phospholipid is PE.

In some examples of the presently disclosed subject matter, the phospholipid moiety comprises PS.

In some examples of the presently disclosed subject matter, the phospholipid moiety comprises PI.

In some examples of the presently disclosed subject matter, the phospholipid moiety comprises PG.

In some examples, the phospholipid is a sphingomyelin. The sphingomyelins consist of a ceramide unit with a phosphorylcholine moiety attached to position 1 and thus in fact is an N-acyl sphingosine. The phosphocholine moiety in sphingomyelin contributes the polar head group of the sphingomyelin.

In some examples, the cannabinoid-lipid conjugate is represented by the general formula (Ia):

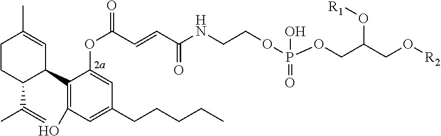

wherein the R₁ and R₂, each represent independently, a saturated or unsaturated acyl, alkyl, alkyl ether or alkenyl chain, or at least one of R₁ and R₂ is a hydrogen.

In some examples of the presently disclosed subject matter, position 2a marked in formula Ia is the position that occupies a hydroxyl group when the cannabinoid (in this specific case CBD) is in its free form.

In some examples of the cannabinoid-lipid conjugate is represented by the general formula (Ia), R₁ and R₂ are identical.

In some examples of the cannabinoid-lipid conjugate is represented by the general formula (Ia), R₁ and R₂ are each an acyl chain.

In some examples of the cannabinoid-lipid conjugate is represented by the general formula (Ia), R₁ and R₂ are each —C(O)—(CH₂)₁₀CH₃ chains.

In some examples of the presently disclosed subject matter, the cannabinoid-lipid conjugate (CBD-MA-DLPE) is represented by the general formula (Ib): 3-((hydroxy (2-((E)-4-(((1′R,2′R)-6-hydroxy-5′-methyl-4-pentyl-2′-(prop-1-en-2-yl) 1′,2′, 3′,4′-tetrahydro-[1,1′-biphenyl]-2-yl) oxy)-4-oxobut-2 enamido) ethoxy) phosphoryl) oxy) propane-1,2-diyl didodecanoate

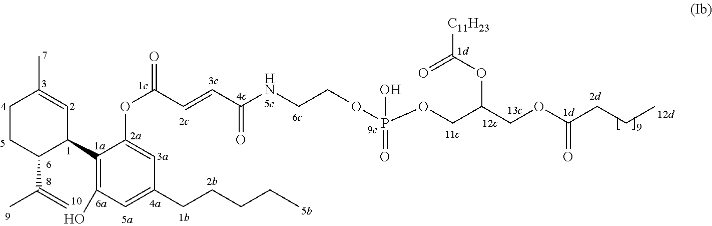

The conjugate, such as that of formula (Ib) can be characterized by full NMR analysis and by mass spectroscopy (MS).

Mass spectra can be conducted for characterizing the conjugate, using LC-MS, ESI, positive ionization.

In addition, Nuclear Magnetic Resonance (NMR) analysis can be conducted for characterizing the conjugate, the conditions of which can be in line with conditions provided hereinbelow in the non-limiting Examples, the conditions forming part of the present disclosure. For example, the NMR spectra can be performed for 1H, 13C and 15N, and 31P, at 278° C., in CDCl₃ containing tetramethylsilane (TMS) as internal reference.

In some examples, the conjugate of formula (Ib) is characterized by at least the following NMR peaks of Table 2 provided below and constituting and integral part of the presently disclosed subject matter.

The presently disclosed subject matter also provides a method for producing the cannabinoid-lipid conjugate disclosed herein.

The method disclosed herein comprises:
(i) reacting the phospholipid (PL) dissolved in an organic solvent with maleic anhydride (MA) to obtain PL-MA intermediate;
(ii) reacting the PL-MA intermediate with a cannabinoid in the presence of a carboxyl activating agent and an esterification agent to obtain a reaction mixture comprising said conjugate.

In some examples of the presently disclosed subject matter, the reaction with maleic anhydride can be conducted in the presence of a base. The base can be an organic base and/or an inorganic base.

In some examples of the presently disclosed subject matter, the base is an organic base.

In some examples of the presently disclosed subject matter, the organic base is selected from the group consisting of pyridine, halo-pyridine, imidazole, N-methylimidazole, triethylamine.

In some examples of the presently disclosed subject matter, the organic base is pyridine.

In some examples of the presently disclosed subject matter, the reaction can be carried out in the presence of inorganic bases.

In some examples of the presently disclosed subject matter, the inorganic base is selected from, without being limited thereto, sodium carbonate, potassium carbonate, sodium bicarbonate, potassium bicarbonate, sodium hydroxide, potassium hydroxide, etc.

In some examples of the presently disclosed subject matter, the ratio between the PL and the MA in this reaction step is essentially equimolar. In this context, when referring to an essentially equimolar ratio it is to be understood to be essentially 1:1, with some deviations, such as a molar ratio of between about 1:0.8 and 1:3, at times, between 1:1 and 1:2, at times, between 1:1 and 1:1.5.

The reaction between the phospholipid and the maleic anhydride provides a PL-MA intermediate. The PL-MA intermediate can be represented by the general formula (III):

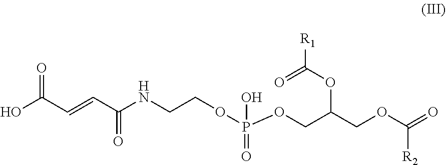

wherein R₁ and R₂ have the meaning as defined hereinabove.

The PL-MA intermediate is then reacted with the cannabinoid.

In some examples of the presently disclosed subject matter, prior to reacting with the cannabinoid, the PL-MA intermediate can be washed with acid and isolated for further and/or other uses.

Non-limiting examples for acids suitable for washing the PL-MA intermediate include inorganic acids, such as HCl (preferably diluted to about 1M) and KHSO₄ (also preferably diluted to about 1M).

In some examples, the PL-MA intermediate, with or without the washing with the acid, is dried prior to the reaction with the cannabinoid.

The reaction of the PL-MA intermediate with the cannabinoid is in the presence of a carboxyl activating agent and an esterification agent.

In the context of the presently disclosed subject matter, when referring to a carboxyl activating agent it is to be understood to encompass coupling reagents for carboxyl groups.

In some examples of the presently disclosed subject matter, carboxyl activating agent is a carbodiimide.

In some examples of the presently disclosed subject matter, the carbodiimide is selected from the group consisting of N-Ethyl-N′-(3-dimethylaminopropyl) carbodiimide hydrochloride (EDC-HCl), dicyclohexylcarbodiimide (DCC), diisopropylcarbodiimide (DIC).

In some examples of the presently disclosed subject matter, the carbodiimide is EDC-HCl.

In some examples of the presently disclosed subject matter, the carboxyl activating agent is a triazole compound. A non-limiting list of triazoles include hydroxybenzotriazole (HOBt), 1-hydroxy-7-azabenzotirazole (HOAt), Cl-HOBt, NO₂-HOBt, CF₃-HOBt, all commonly used as coupling additives to increase reactivity of leaving groups.

In some examples of the presently disclosed subject matter, the carboxy activating agent is an HOBt-based aminium/phosphonium salt, including, without being limited thereto, benzotriazol-1-yloxytris (dimethylamino) phosphonium hexafluorophosphate (BOP), benzotriazol-1-yloxytripyrrolidinophosphonium hexafluorophosphate (PyBOP), 2-(1H-benzotriazol-1-yl)-1,1,3,3-tetramethyluronium hexafluorophosphate (HBTU), 1-[Bis (dimethylamino) methylene]-1H-1,2,3-triazolo[4,5-b]pyridinium 3-oxide hexafluorophosphate (HATU), O-(1H-6-Chlorobenzotriazole-1-yl)-1,1,3,3-tetramethyluronium hexafluorophosphate (HCTU).

In the context of the presently disclosed subject matter, when referring to an esterification agent it is to be understood to have its common meaning in the art when using the Steglich Esterification reaction.

In some examples of the presently disclosed subject matter, the esterification agent is an organic base. In some examples, the organic base is selected from the group consisting of 4-Dimethylaminopyridine (DMAP), triethylamine (Et3N), N-Methylmorpholine (NMM), pyridine, N,N-Diisopropylethylamine (DIPEA), imidazole.

In some examples of the presently disclosed subject matter, the esterification agent is DMAP.

In some examples of the presently disclosed subject matter, the reaction between the PL-MA and the cannabinoid is under reflux conditions.

The resulting cannabinoid-lipid conjugate can be purified, using any technique known in the art.

In some examples of the presently disclosed subject matter, the resulting conjugate is purified and lyophilized.

In some examples of the presently disclosed subject matter, the herein disclosed method is used for the production of CBD-MA-DLPE conjugate of formula (Ib), the method being schematically illustrated in the Scheme (1) below:

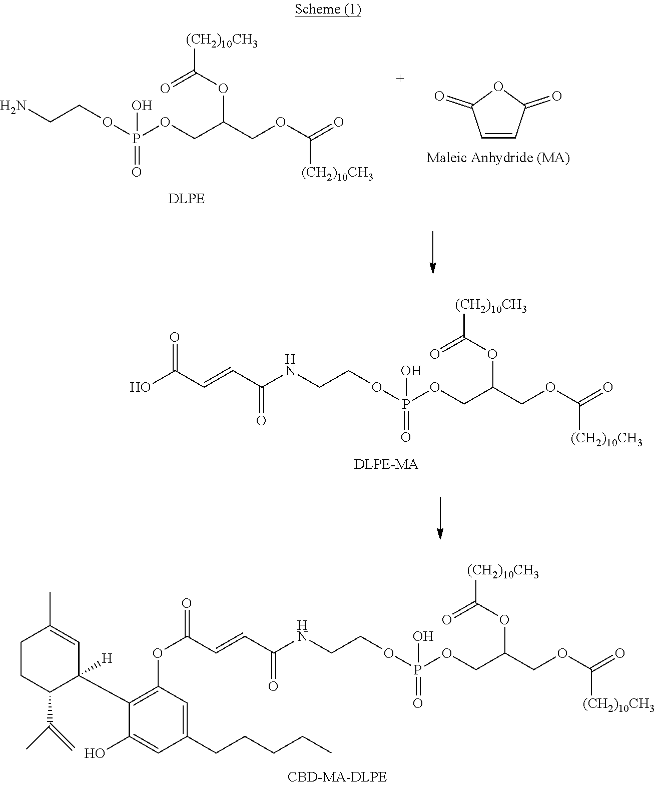

The cannabinoid-lipid conjugate can be used for the delivery and release of the cannabinoid once within a target body, e.g. within a subject's body or even at a target site.

Without being bound thereto, tt has been found that the A-MA linkage (in A-MA-PL conjugate) is cleavable in the presence of enzymes such as, an esterase.

The esterase can be any member of the family of esterase enzyme.

In some examples of the presently disclosed subject matter, the esterase is a carboxylesterase (also known by the term carboxylic-ester hydrolase).

In some examples of the presently disclosed subject matter, the enzyme is any member of the enzymes that fall under the family of carboxylesterase, these include carboxylesterase 1 (CES1), carboxylesterase 2 (CES2), carboxylesterase 3 (CES3), and others.

Thus, once at proximity with the enzyme, the cannabinoid is ‘freed’ from its linkage to the phospholipid carrier by the enzymatic action.

In some examples of the presently disclosed subject matter, the cannabinoid-lipid conjugate disclosed herein can be integrated into lipid membranes, where the hydrophobic tail(s) of the phospholipid is at least partially embedded (anchored) into the lipid membrane. Thus, in some examples of the presently disclosed subject matter, the cannabinoid-lipid conjugate is anchored onto a lipid-based particle (e.g. nanoparticle), in a non-covalent manner.

In the context of the present disclosure, when referring to a “lipid-based particle”, it is to be understood to encompass any nano or micron-sized particle having an external lipid membrane.

In some examples of the presently disclosed subject matter, the particle is a nanoparticle. The lipid membrane can be a monolayer, a lipid bilayer, oligolamellar as well as multilamellar type vesicles.

The cannabinoid-lipid conjugate, either in free form (i.e. unbound to a nanoparticle) or in association with a delivery vehicle, e.g. particle can be formulated with a physiologically acceptable carrier to form an administrable composition. The composition can be, without being limited thereto, a pharmaceutical composition, a cosmetic composition, a nutraceutical composition, a diagnostic composition etc.

In relation to the above, there is thus provided in accordance with the presently disclosed subject matter a method for delivering cannabinoid to a target site, the method comprises administering to a target body including said target site an amount of the presently disclosed cannabinoid-phospholipid conjugate.

In the context of the presently disclosed method, it is to be understood that the target body can include any media in which the release of a free cannabinoid is desired. The media can be an in vitro media, e.g. in diagnostic methods, or a living body, e.g. animal body, where the release and delivery of the free cannabinoid at a target organ, tissue or cell is desired.

In some examples, the target body is a mammal body. In some examples, the target body is the human body.

In some examples, the presently disclosed subject matter provides a method of treatment, that involves administering to a subject (e.g. mammalian) in need of treatment and amount of the presently disclosed cannabinoid-phospholipid conjugate.

The conjugate either in free form or in association with a delivery vehicle, can be administered and dosed in accordance with good medical practice, taking into account the clinical condition of the individual in need thereof, the site and method of administration, scheduling of administration, patient age, sex, body weight and other factors known to medical practitioners.

The amount of the conjugate will be an effective amount. The term “effective amount” for purposes herein is thus determined by such considerations as are known in the art. The amount must be effective to achieve the desired effect from the cannabinoid.

The conjugate can be combined with pharmaceutically acceptable carriers, diluents, excipients, additives and adjuvants, as known in the art.

The conjugate can be administered by any means known in the art, including, without being limited thereto, intra-abdominal, intra-amnionic, intra-arterial, intra-articular, intra-biliary, intra-cardiac, intra-cartilaginous, intra-caudal, intra-cavernous, intra-cerebral, intra-cisternal, intra-corneal, intra-coronal, intra-coronary, intra-corporus cavernosum, intra-dermal, intradiscal, intra-ductal, intra-duodenal, intra-dural, intra-epidermal, intra-esophageal, intra-gastric, intra-gingival, intra-ileal, intra-lesional, intra-lymphatic, intra-medullary, intra-meningeal, intra-muscular, intra-ocular, intra-ovarian, intra-pericaridal, intra-peritoneal, intra-pleural, intra-prostatic, intra-pulmonary, intra-sinal, intra-spinal, intra-synovial, intra-tendinous, intra-testicular, intra-thecal, intra-thoracic, intra-tubular, intra-tumor, intra-tympanic, intra-uterine, intra-vascular, intra-venous, administration as well as infusion techniques.

As used herein, the forms “a”, “an” and “the” include singular as well as plural references unless the context clearly dictates otherwise. For example, the term “cannabinoid” includes one or more cannabinoids.

Further, as used herein, the term “comprising” is intended to mean that the recited elements but not excluding other elements. The term “consisting essentially of” is used to define the recited elements but exclude other elements that may have an essential significance on essence of the disclosed subject matter. “Consisting of” shall thus mean excluding more than trace elements of such other elements. Embodiments defined by each of these transition terms are within the scope of this invention.

Further, all numerical values, e.g. when referring the amounts or ranges of the elements constituting the disclosed subject matter are approximations which are varied (+) or (−) by up to 20%, at times by up to 10% of from the stated values. It is to be understood, even if not always explicitly stated that all numerical designations are preceded by the term “about”

The invention will now be described by way of non-limiting examples that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described hereinbelow.

### SOME NON-LIMITING EXAMPLES

### Example 1A-Synthesis of CBD-MA-DLPE conjugate

In the following examples, the procedure for synthesizing the cannabinoid-PL conjugate follows the Scheme provided above and involved connecting the DLPE to maleic anhydride to form DLPE-MA and then the coupling of the DLPE-MA to the CBD to form the conjugate. The PL connection to maleic anhydride was in an organic solvent with addition of an organic base. The second coupling of PL-MA approach is performed with any cannabinoid such as CBD, CBG, CBN, CBDV, CBDA, THC and any functional analogues thereof. In the non-limiting example below, the PL-MA was conjugated to CBD can be used on any other cannabinoid (CBD, CBG, CBN, CBDV, THC etc. and any functional analogues thereof)

### Materials

Table 1 provides the materials used in the non-limiting example:

TABLE 1
list of materials

| Material | Manufacturer |
|---|---|
| 12:0 PE - 1,2-Dilauroyl-sn-glycero-3- | Avanti |
| phosphorylethanolamine | |
| Maleic anhydride | Glentham Life science |
| Pyridine | Sigma-Aldrich |
| DMAP | TCI |
| EDC-HCl (N-(3-Dimethylaminopropyl)-N′- | TCI |
| ethylcarbodiimide hydrochloride) | |
| CBD (Canapure PH) | Symrise |
| Ammonium hydrogen carbonate | MercK |
| Ammonium hydroxide sol. ≥25% in H₂O | Fluka |
| Trifluoroacetic acid (TFA) | BIoLab |
| Acetonitrile | Bio-Lab/Merck |
| Iso-propanol (IPA) | Bio-Lab |
| Water | Bio-Lab |
| Chloroform | BioLab |
| Methylene chloride (DCM) | BioLab |

### Methods

Liquid Chromatography-Mass Spectrometry (LC-MS) analytical method was conducted using LC-MS, ESI, positive ionization.

NMR, was conducted using the following conditions:
Bruker Avance-III-700 instrument (700.5, 176.1 and 71.0 MHz for ¹H ¹³C and ¹⁵N, respectively). All spectra were taken at 278° K, in CDCl₃ containing tetramethylsilane (TMS) as internal reference. In order to facilitate signal assignment and to prove connectivity, several 2D experiments were also performed including: COSY (through-bond ¹Hx¹H correlation), TOCSY (total ¹Hx¹H correlation), HMQC (one-bond ¹Hx¹³C correlation), HMBC (long-range ¹Hx¹³C correlation) and HMBC-N(long-range ¹Hx¹⁵N correlation). The 31P spectrum was taken in a Bruker Avance-II+-500 instrument, at 202.4 MHz.

### Synthetic Pathway

(E)-4-((2-(((2,3-bis (dodecanoyloxy) propoxy) (hydroxy) phosphoryl) oxy) ethyl) amino)-4-oxobut-2-enoic acid

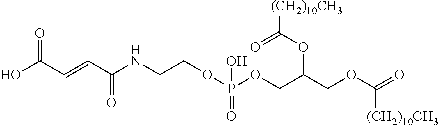

Into 250 ml flask DLPE (752 mg, 1.3 mmol, leq) in 181 ml chloroform was added. To the white solution pyridine 15 ml and maleic anhydride (127 mg, 1.3 mmol, leq) were then added. The reaction turned to colorless solution and left to stir while monitoring overnight.

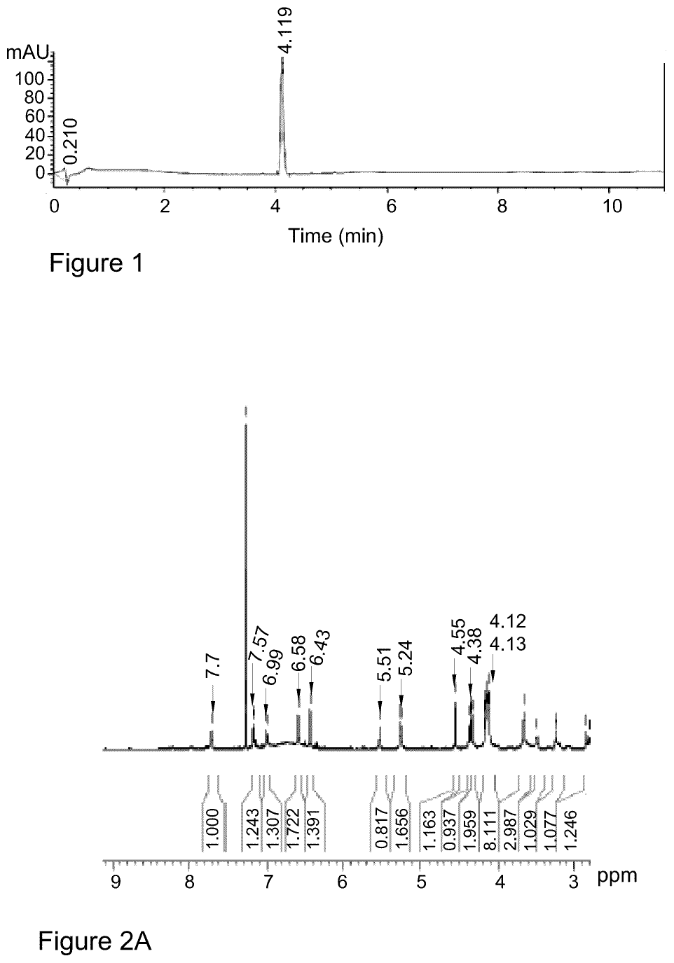

The resulting DLPE-MA was analyzed by LC-MS, as shown in FIG. 1. Specifically, under the conditions employed, there was obtained a clear peak. According to LC-MS, there was a fit mass 678 [M+H]⁺.

According to TLC (not shown), no DLPE remained in the mixture.

The reaction mixture was washed with HCl IM (150 ml) and then dried over Na₂SO₄, evaporated to dryness, to give 908 mg of DLPE-MA (>99% yield).

### 3-((hydroxy (2-((E)-4-(((1′R,2′R)-6-hydroxy-5′-methyl-4-pentyl-2′-(prop-1-en-2-yl) 1′,2′, 3′, 4′-tetrahydro-[1,l′-biphenyl]-2-yl) oxy)-4-oxobut-2 enamido) ethoxy) phosphoryl) oxy) propane-1,2-diyl didodecanoate

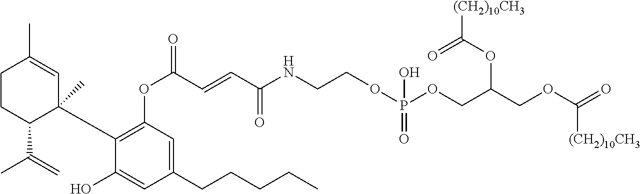

Into 250 ml flask CBD (384 mg, 1.22 mmol, 0.9 eq), DLPE-MA (908 mg, 1.34 mmol, 1 eq) in 70 ml dichloromethane (DCM) were added.

The reaction was cooled to 0° C. and EDC-HCl (12.7 mg, 0.062 mmol, 1.12 eq) and DMAP (164 mg, 1.34 mmol, leq) were added. The mixture was allowed to warm to room temperature, while being monitored by LC-MS and TLC. Additional reflux for three days was performed, and the solvent was evaporated to give a crude product which was further purified on preparative HPLC. The purified fractions were dried to give 282 mg (21% yield) of pure product.

The product was analyzed by LC-MS and NMR.

The mass identification was obtained on a negative mode and was shown to be m/z: 972.5 [M−H]⁻.

The NMR data is provided in Table 2.

TABLE 2
NMR Data

| | ¹H | ¹³C | | ¹H | ¹³C (¹⁵N, ³¹P) |
|---|---|---|---|---|---|
| 1 | 3.48 | 37.68 | 1b | 2.50 | 35.26 |
| 2 | 5.51 | 122.92 | 2b | 1.58 | 30.55 |
| 3 | — | 140.69 | 3b | 1.27 | 31.44 |
| 4 | 1.69, 1.78 | 27.82 | 4b | 1.27 | 22.51 |
| 5 | 2.12, 2.22 | 30.20 | 5b | 0.87 | 14.07 |
| 6 | 2.43 | 45.67 | 1c | — | 163.99 |
| 7 | 1.77 | 23.68 | 2c | 6.99 | 129.62 |
| 8 | — | 146.69 | 3c | 7.17 | 137.62 |
| 9 | 4.38, 4.55 | 111.85 | 4c | — | 164.09 |
| 10 | 1.56 | 19.71 | 5c | 7.70 | (−262.6)^{b} |
| 1a | — | 118.78 | 6c | 3.23, 3.65 | 40.10[6]^{a} |
| 2a | — | 148.75 | 7c | 4.13 | 65.97 [5]^{a} |
| 3a | 6.43 | 113.58 | 9c | — | (0.17)^{c} |
| 4a | — | 142.94 | 11c | 4.12 | 64.95[7]^{a} |
| 5a | 6.58 | 114.85 | 12c | 5.24 | 69.40[7]^{a} |
| 6a | — | 155.61 | 13c | 4.16, 4.34 | 61.91 |
| 1d | — | 173.38^{d}, 173.68^{e} | | | |
| 2d | 2.32 | 33.98, 34.14 | | | |
| 3d | 1.58 | 24.79, 24.82 | | | |
| 4d-7d | 1.2-1.3 | 29.4 ± 0.3 | | | |
| 8d | 1.2-1.3 | 31.91 | | | |
| 9d | 1.2-1.3 | 22.70 | | | |
| 10d | 0.87 | 14.17 | | | |

^{a}in square brackets J_{CP} in Hz;
^{b15}N;
^{c31}P
^{d}carbonyl connected to C_{12c};
^{e}carbonyl connected to C_{13c}

The obtained compound was then determined to have the following structure:

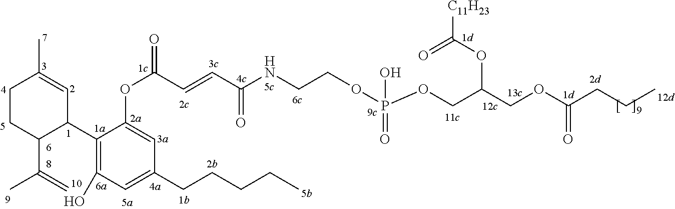

### Results

The results show an effective process for producing DLPE-MA-CBD conjugate with 21% yield and at more than 95% HPLC purity.

### Example 1B-Synthesis of CBD-MA-DLPE conjugate

The above synthesis procedure was repeated under the same conditions, with slight variations as follows:

Into 100 ml Round Bottom Flask (RBF) was added MA-DLPE (2.5 gr, 3.71 mmol) and CBD (1.16 gr, 3.71 mmol) in 30 ml anhydrous dichloromethane. Trimethylamine (0.96 ml, 7.42 mmol), EDC (1 gr, 5.57 mmol) and DMAP (45 mg) were added to the solution. The reaction mixture was heated at reflux for overnight under inert atmosphere.

The reaction mixture was then quenched and extracted with 1M HCl, and then the organic phase was washed with saturated sodium bicarbonate solution, dried over sodium sulfate, filtered and evaporated.

The crude CBD-MA-DLPE was purified on silica gel column chromatography using DCM and MeOH as the eluent (Gradient from 0% MeOH up to 20% MeOH in DCM).

The desired CBD-MA-DLPE product was obtained as solid powder after evaporation and analyzed.

The CBD-MA-DLPE product was then analyzed by HPLC, under the following conditions

HPLC column: Luna Omega 3 μm polar C18 100° A, 150×4.6 mm

Column temperature: 30° C.

Flow rate: 1 ml/min

UV detection: 220 nm

Mobile phase A: 10 mM ammonium carbonate

Mobile phase B: acetonitrile

The mobile phase gradient program was as follows:

| Time (min) | Solution A % | Solution B % |
|---|---|---|
| 0 | 75 | 25 |
| 0.5 | 75 | 25 |
| 4.5 | 10 | 90 |
| 10.0 | 10 | 90 |
| 11.0 | 5 | 95 |
| 14.0 | 5 | 95 |
| 16.0 | 75 | 25 |

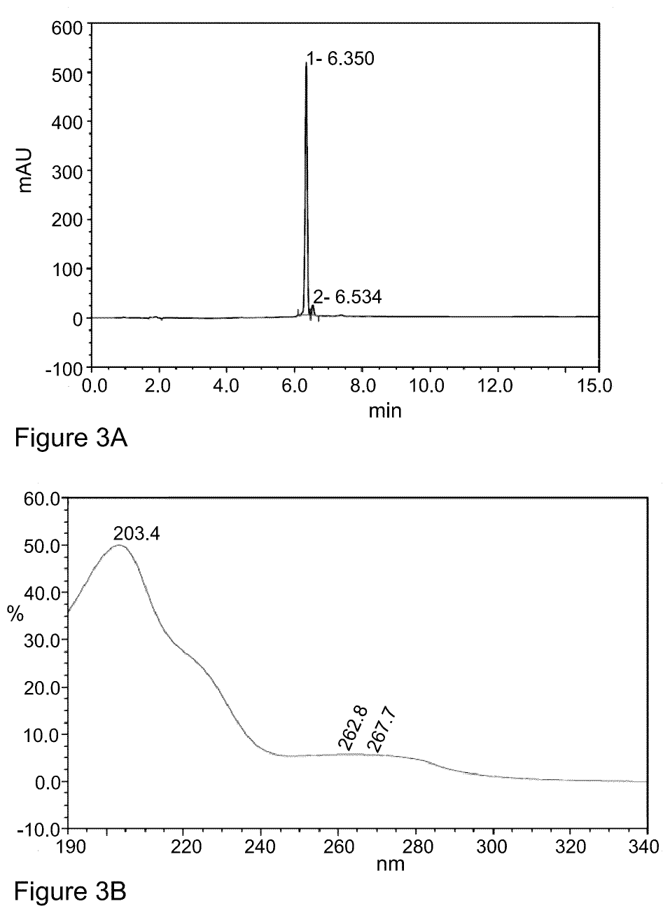

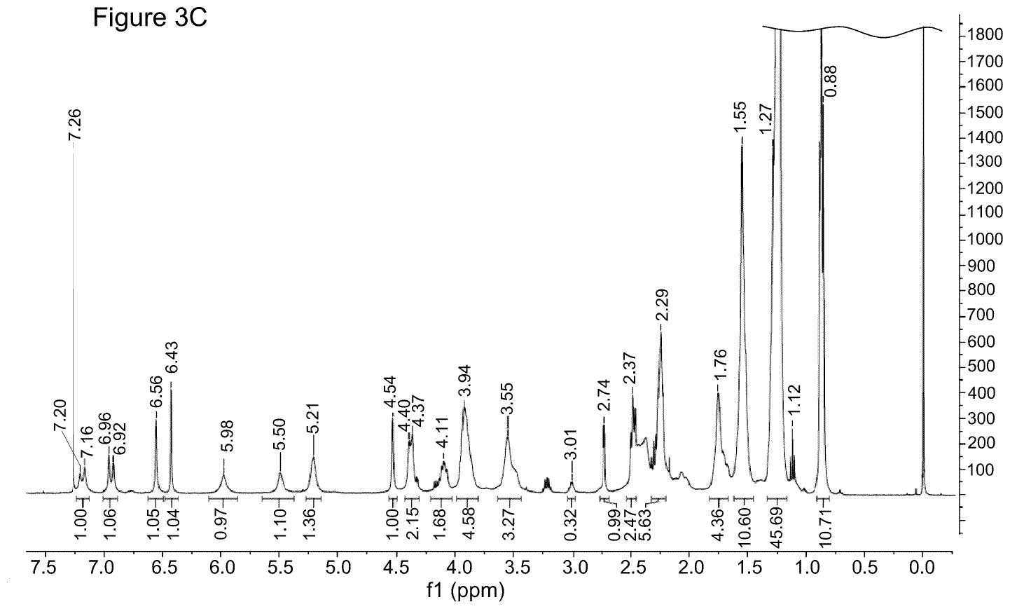

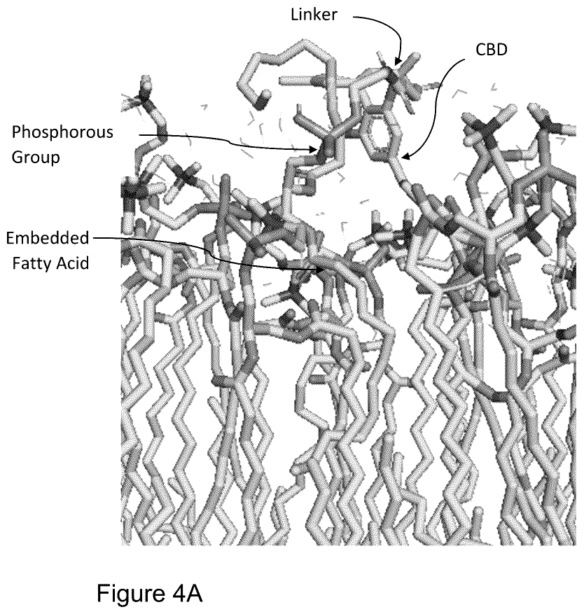

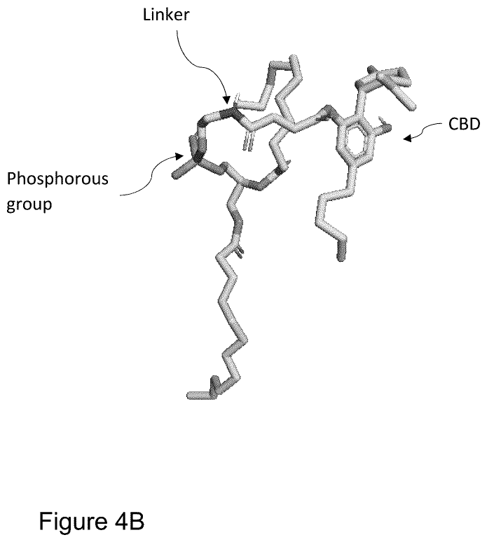

The HPLC results are provided in FIG. 3A.

In addition, FIG. 3B provides the UV absorbance of the resulting CBD-MA-DLPE product and FIG. 3C provides the H-NMR (400 MHz) spectrum.

¹H-NMR (400 MHZ, CDCl₃), d: 7.18 (1H, d), 6.96 (1H, d), 6.56 (1H, s), 6.43 (1H, s), 5.98 (1H, s), 5.50 (1H, s), 5.21 (1H, s), 4.54 (1H, s), 4.40 (2H, m), 4.11 (1H, m), 3.94 (4H, m), 3.55 (2H, m), 2.73 (1H, d), 2.73 (2H, m), 2.29 (4H, m), 1.76 (4H, m), 1.55 (12H, m), 1.27 (36H, m), 0.87 (9H, m).

The above synthesis yielded 0.7 gr product (about 15% yield, mass of 972 ESI, r.t (product)=6.3 min (purity >95%)).

A computational presentation of the resulting DLPE-MA-CBD conjugate in accordance with Examples 1A-1B is provided in FIGS. 3A-3B provide. These presentations illustrate the phosphorous group to which the two fatty acid chains are linked, the cleavable linker and the CBD group (FIG. 3A) and the spatial configuration of the conjugate, similarly pointing at the phosphorous group to which the two fatty acid chains are linked, the cleavable linker and the CBD group (FIG. 3B).

### Example 2-Synthesis of DLPE-MA-CBN conjugate

The synthesis of DLPE-MA-CBN conjugate follows the following reaction scheme:

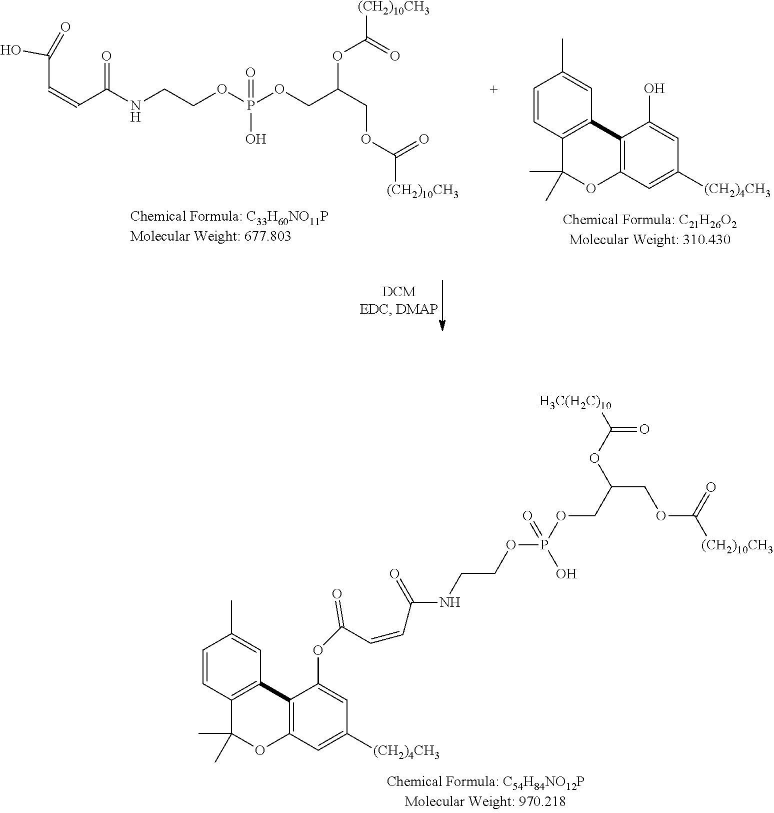

The synthesis included the following steps:

Into 100 ml RBF was added MA-DLPE (1.66 gr, 2.46 mmol) and CBN (0.77 gr, 2.46 mmol) in 20 ml anhydrous dichloromethane. EDC (0.46 gr, 2.95 mmol) and DMAP (0.45 gr, 3.69 mmol) were added to the solution. The reaction mixture was heated at reflux for 2 hours under inert atmosphere.

The reaction mixture was then quenched and extracted with 1M HCl, and then the organic phase was washed with 5% sodium bicarbonate solution, dried over sodium sulfate, filtered and evaporated.

The crude was purified on silica gel column chromatography using DCM and MeOH as the eluent (Gradient from 0% MeOH up to 20% MeOH in DCM).

The desired product was obtained as yellow oil after evaporation. (0.67 gr, about 30% yield, mass of 969 ESI-) r.t (product)-6.64 min, purity >95%)

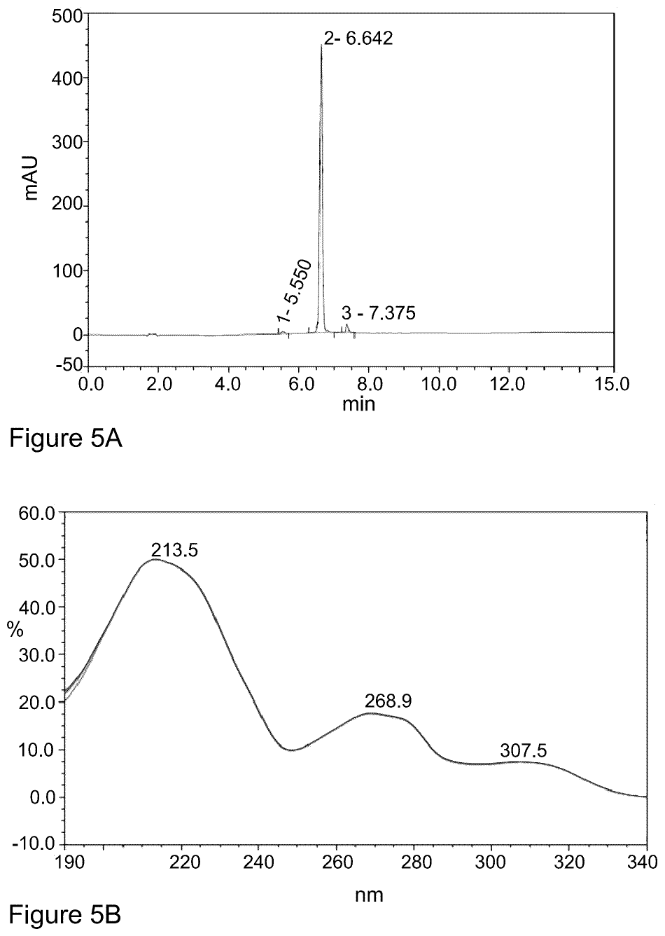

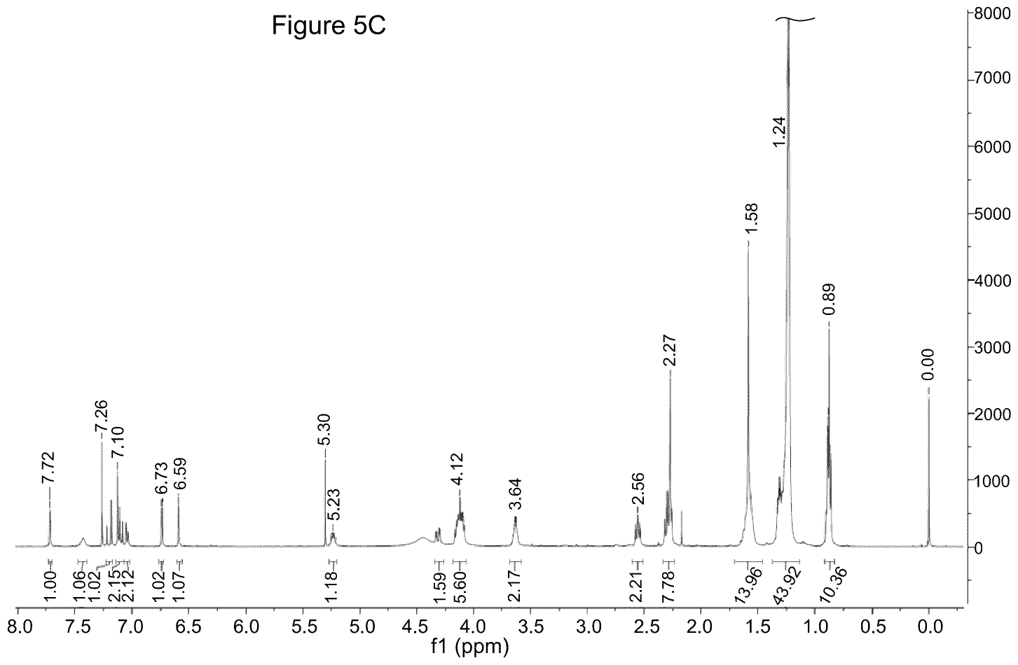

The DLPE-MA-CBN product was then analyzed by HPLC, under the conditions described above with respect to Example 1B, and is provided in FIG. 5A. In addition, FIG. 5B provides the UV absorbance of the resulting DLPE-MA-CBN product and FIG. 5C provides the H-NMR (400 MHz) spectrum.

1H-NMR (400 MHZ, CDCl₃), d: 7.72 (1H, s), 7.43 (1H, bs), 7.18 (1H, d), 7.09 (2H, m), 7.04 (1H, d), 6.73 (1H, s), 6.59 (1H, s), 5.23 (1H, m), 4.40 (1H, bs), 4.30 (1H, dd), 4.11 (5H, m), 3.64 (2H, m), 5.56 (2H, dd), 2.30 (7H, m), 1.58 (12H, m), 1.29 (36H, m), 0.89 (9H, m). MS (ESI-) m/z: 969. r.t (product)-6.64 min, purity >95%).

### Example 3-Synthesis of DLPE-MA-CBG conjugate

The synthesis of DLPE-MA-CBG conjugate follows the following reaction scheme:

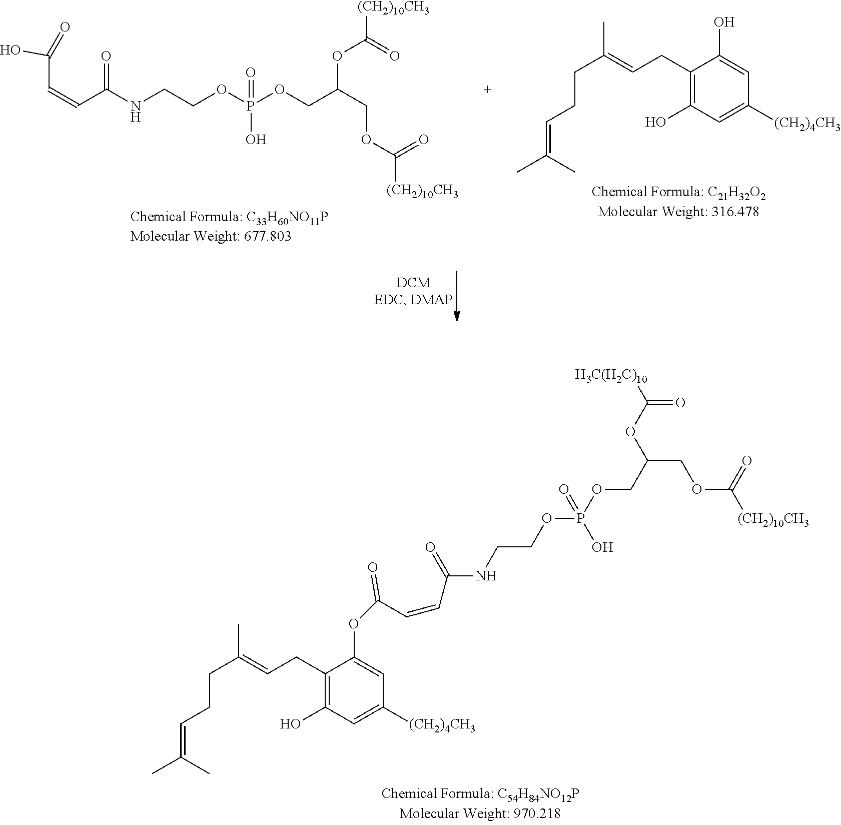

The synthesis included the following steps:

Into 100 ml RBF was added MA-DLPE (1.66 gr, 2.46 mmol) and CBG (0.78 gr, 2.46 mmol) in 20 ml anhydrous dichloromethane. EDC (0.46 gr, 2.95 mmol) and DMAP (0.45 gr, 3.69 mmol) were added to the solution. The reaction mixture was heated at reflux for 2 hours under inert atmosphere.

The reaction mixture was then quenched and extracted with 1M HCl, and then the organic phase was washed with 5% sodium bicarbonate solution, dried over sodium sulfate, filtered and evaporated.

The crude DLPE-MA-CBG was purified on silica gel column chromatography using DCM and MeOH as the eluent (Gradient from 0% McOH up to 20% MeOH in DCM).

The desired DLPE-MA-CBG product was obtained as yellow oil after evaporation. (0.67 gr, about 30% yield, mass of 975 ESI-) r.t (product)-6.21 min, purity >95%)

1-42. (canceled)

43. A cannabinoid-phospholipid conjugate having the general formula (I): 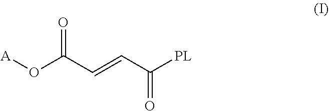
wherein A is a cannabinoid and PL is a phospholipid (PL); and
wherein the cannabinoid (A) is covalently linked, via a cleavable linker, to a polar head group of the PL.

44. The conjugate of claim 43, wherein said cannabinoid (A) is linked to said linker at position C2a thereof according to a numbering in free cannabidiol (CBD) of formula (II):

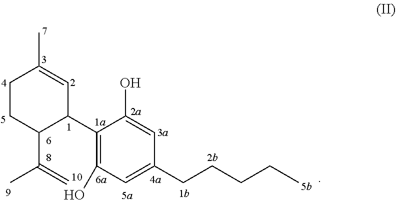 [SMILES: C=C(C)C1CCC(C)=CC1c1c(O)cc(CCCCC)cc1O]

45. The conjugate of claim 43, wherein said cannabinoid (A) is linked at position C6a of thereof according to a numbering in free cannabidiol (CBD) of formula II:

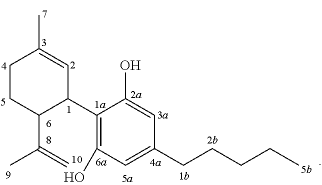 [SMILES: C=C(C)C1CCC(C)=CC1c1c(O)cc(CCCCC)cc1O]

46. The conjugate of claim 43, wherein said cannabinoid is cannabidiol (CBD) or CBD analogue selected from the group consisting of cannabidiol (CBD), cannabidiolic acid (CBDA), cannabidiol-dimethylheptyl (CBD-DNH), cannabidivarin (CBDV), cannabidivarinolic acid (CBDVA), cannabidiolic acid (CBDA), cannabidiol monomethyl ether (CBDM), cannabidiolquinones (CBDQ), Cannabidiol hydroxy quinone (CBDHQ), and abnormal CBD (Abn-CBD).

47. The conjugate of claim 43, wherein said cannabinoid is tetrahydrocannabinol (THC) or THC functional analogue selected from the group consisting of delta9-THC, delta8-THC, trans-DELTA 10-tetrahydrocannabinol (trans-DELTA 10-THC), cis-DIO-tetrahydrocannabinol (cis-DELTA10-THC), tetrahydrocannabinolic acid C4 (THCA-C4), tetrahydrocannbinol C4 (THC-C4), tetrahydrocannabivarinic acid (THCVA), tetrahydrocannabivarin (THCV), DELTA8-tetrahydrocannabivarin (DELTA8-THCV), DELTA9-tetrahydrocannabivarin (DELTA9-THCV), Delta-9-tetrahydrocannabinolic acid B (DELTA9-THCA-B), tetrahydrocannabiorcolic acid (THCA-C1), tetrahydrocannabiorcol (THC-C1), DELTA7-cis-iso-tetrahydrocannabivarin, DELTA8-tetrahydrocannabinolic acid (DELTA8-THC-A), DELTA9-tetrahydrocannabinolic acid (DELTA9-THC-A), 11-hydroxy-DELTA9-tetrahydrocannabinol (11-OH-THC), 11-nor-9-carboxy-DELTA9-tetrahydrocannabinol, 10 ethoxy-9-hydroxy-DELTA6a-tetrahydrocannabinol, 10-oxo-DELTA6a (10a)-tetrahydrocannabinol (OTHC), DELTA9-cis-tetrahydrocannabinol (cis-THC), trihydroxy-delta-9-tetrahydrocannabinol (triOH-THC), Delta-9-tetrahydrocannabivarinic acid (THCVA), 10-Oxo-delta-6a-tetrahydrocannabinol (OTHC), isotetrahydrocannabinol (iso-THC).

48. The conjugate of claim 43, wherein said cannabinoid is a CBN or CBN functional analogue selected from the group consisting of cannabinol (CBN), cannabinolic acid (CBNA), cannabinol methyl ether (CBNM), cannabinol C4 (CBN-C4), cannabinol C2 (CBN-C2), cannabinol C1 (CBN-C1), and cannabinodiol (CBND).

49. The conjugate of claim 43, wherein said PL has the general formula (II): 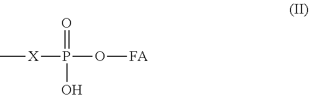
wherein
FA represents a glycerol backbone or a sphinogosine backbone carrying one or two acyl, alkyl or alkenyl chains, which may be the same or different;
X represents an oxygen or a polar head group to which said cleavable linker is bound.

50. The conjugate of claim 43, wherein said polar group is selected from phosphatidyl serine (PS), phosphatidylethanolamine (PE), phosphatidylcholine (PC), phosphatidylglycerol (PG), phosphatidylinositol (PI).

51. The conjugate of claim 49, wherein said X is oxygen.

52. The conjugate of claim 43, wherein said PL is PE and said cannabinoid A is CBD.

53. The conjugate of claim 43, having the general formula (Ia): 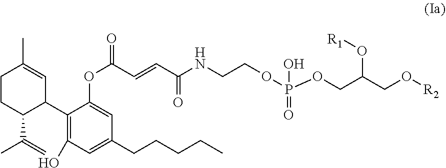
wherein said R₁ and R₂, each represent independently, a saturated or unsaturated acyl, alkyl, alkyl ether or alkenyl chain, or at least one of R₁ and R₂ is a hydrogen.

54. The conjugate of claim 53, wherein said R₁ and R₂ are identical.

55. The conjugate of claim 53, wherein said R₁ and R₂ are each an acyl chain.

56. A method for obtaining a cannabinoid-phospholipid conjugate having the general formula (I): 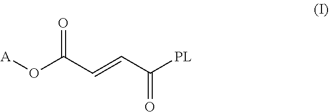
wherein A is a cannabinoid and PL is a phospholipid (PL); and
wherein the cannabinoid (A) is covalently linked, via a cleavable linker, to a polar head group of the PL,
the method comprising:
(i) reacting the phospholipid (PL) dissolved in an organic solvent with maleic anhydride (MA) to obtain PL-MA intermediate; and
(ii) reacting said PL-MA intermediate with the cannabinoid in the presence of a carboxyl activating agent and an esterification agent to obtain a reaction mixture comprising said conjugate.

57. The method of claim 56, wherein said reacting of the phospholipid (PL) with maleic anhydride (MA) is in the presence of a base.

58. The method of claim 56, comprising washing said PL-MA intermediate with an acid before reacting same with the cannabinoid.

59. The method of claim 58, wherein said acid comprises HCl or KHSO₄.

60. The method of claim 56, wherein said carboxyl activating agent is a carbodiimide.

61. The method of claim 56, wherein said esterification agent is selected from the group consisting of 4-Dimethylaminopyridine (DMAP), triethylamine (Et₃N), N-Methylmorpholine (NMM), pyridine, N,N-Diisopropylethylamine (DIPEA), imidazole and combinations of same.

62. A method of treatment, comprising administering to a subject in need of treatment and amount of a cannabinoid-phospholipid conjugate according to claim 43.
